(19) United States
(12) Patent Application Publication
(10) Pub. No.: US 2013/0109879 A1
(43) Pub. Date: May 2, 2013

(54) Process for converting natural oils to surfactants and biofuels

(71) Applicants: Paul D. Berger, Sugar Land, TX (US); Christie H. Berger, Sugar Land, TX (US)

(72) Inventors: Paul D. Berger, Sugar Land, TX (US); Christie H. Berger, Sugar Land, TX (US)

(73) Assignee: OIL CHEM TECHNOLOGIES, INC., Sugar Land, TX (US)

(21) Appl. No.: 13/507,416

(22) Filed: Jun. 26, 2012

Related U.S. Application Data
(60) Provisional application No. 61/628,250, filed on Oct. 27, 2011.

(51) Int. Cl. C07C 67/24; C07C 315/04

(52) U.S. Cl. 560/12; 560/11; 560/129

(57) ABSTRACT

A process for converting naturally occurring fats and oils to sulfonated surfactants that can be used in a variety of applications where surfactants are found to be applicable including but not restricted to Enhanced Oil Recovery, paper and pulp processing, mining, metal treating, adhesives, coatings, pesticide formulations, herbicide formulations, fungicide formulations. The byproduct of such a process is a useful solvent or source of biodiesel fuel.

### CROSS REFERENCE TO RELATED APPLICATIONS

This application is based on provisional application 61/628,250 filed on Oct. 27, 2011.

### STATEMENT REGARDING FEDERALLY SPONSORED RESEARCH OR DEVELOPMENT

Not Applicable

### DESCRIPTION OF ATTACHED APPENDIX

Not Applicable

### DESCRIPTION OF FIGURES

No figures are attached

### BACKGROUND OF INVENTION

The supply of petroleum derived fuels has been shown to be rapidly decreasing. This is not because of the lack of sources for oil and gas but because of the limitations of existing methods of primary and secondary oil recover to efficiently remove oil and gas from existing reservoirs. Only about 30 percent of the oil in most reservoirs is removed before they are abandoned. Recently alternatives to petroleum based fuels have been proposed and implemented in limited cases. These include the conversion of certain agricultural crops to alcohols and esters. In many cases the use of these crops to produce fuel competes with their use as food. In most cases the efficiency of these bio-based fuels is not as great as petroleum based fuels. They require a great deal of energy to cultivate, protect from disease, harvest and convert to fuel. If a method to use these bio-based materials to recovery petroleum based products efficiently the amount required would be much less since it has been demonstrated that 1 barrel of bio-based surfactant can recover more than 60 barrels of oil whereas one barrel of bio-based fuel has the energy equivalent of less than 1 barrel of petroleum based fuel.

### BRIEF DESCRIPTION OF INVENTION

This invention involves the conversion of natural oils including, but not limited to those derived from various agricultural seed crops, roots, animal fat, marine fats and other sources to surfactants useful in a variety of applications including but not limited to oilfield, Improved Oil Recovery (10R), Enhanced Oil Recovery (EOR), Mining, Metal Treatment, Paper and Pulp Processing, lubricants, detergents, cleaners. In many cases a useful byproduct of the process will be materials suitable for many uses including but not limited to naturally derived fuel such as biodiesel, and naturally derived solvent.

One of the objects of the present invention is to provide a method of converting the unsaturated portion of naturally occurring glycerides into useful surfactants.

Another objective of the invention is to make surfactant and bio-fuels directly from the vegetable oil

Another object of the present invention is to use the surfactants derived from the invention to recover additional oil through various Improved Oil Recovery (IOR), or Enhanced Oil Recovery (EOR) processes.

Another object of the present invention is to provide a process where the byproduct of making the surfactant is used as an energy source.

Other objects and advantages of the invention will be shown in the discussion and examples below.

### DETAILED DESCRIPTION OF THE INVENTION

The present invention involves converting vegetable oil to a new surfactant and biodiesel. The oil is first reacted with methanol or other low molecular weight alcohols to form the esters of the fatty acids and glycerin. The glycerin formed as a by-product may be recovered by methods known to the art or may be left in the product. The unsaturated methyl esters are than reacted with an aryl alkyl sulfonic acid as described in U.S. Pat. No. 6,043,391 under conditions described in U.S. Pat. No. 7,863,476 to form the new surfactant. Saturated fatty acid I esters present do not react with the aryl alkyl sulfonic acid and they can be easily separated by distillation or extraction because of the boiling point difference between them and the new surfactants formed. These saturated fatty acid esters can be used as bio-fuel or solvent. They can also be left with the surfactant as solvent. The biodiesel formed can also be used to provide energy to run the manufacturing plant producing the surfactant. The surfactants formed through the present invention can be used in a variety of applications where surfactants are found to be applicable including but not restricted to Enhanced Oil Recovery, paper and pulp processing, mining, metal treating, adhesives, coatings, pesticide formulations, herbicide formulations, fungicide formulations.

The present invention includes the sequence of reactions below:

Step 1: A glyceride (1) is first trans esterified with alcohol (2) to form glycerin (3) and the esters (4) of the acids formed through transesterification.

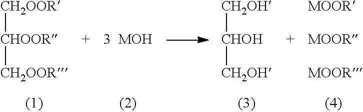

where R, R′, R″ are alkyl or alkenyl but at least one is alkenyl,

M=C1-6 alkyl.

### FIG. 1 Trans Esterification of the Glyceride

Step 2: The unsaturated esters or fatty acids derived from the glycerides will react with the aryl alkyl sulfonic acid (5) to form a new surfactant combining both the aryl alkyl sulfonic acid and the fatty acid ester into one new anionic surfactant molecule (6). This is subsequently neutralized with alkali or amines to form the salt of the aryl alkyl sulfonic acid (7).

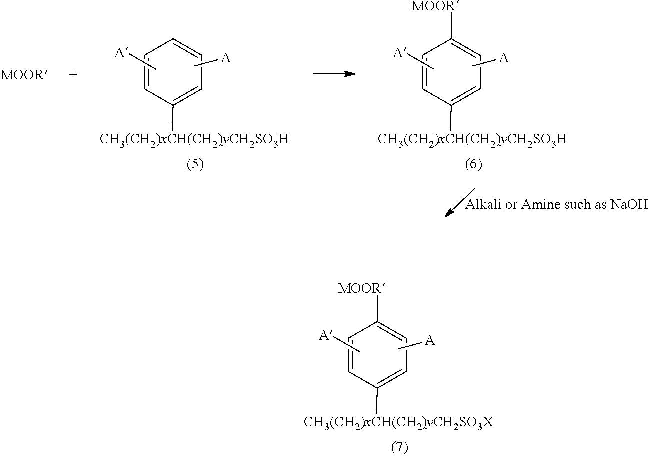

Where;

R, R′, R″ are alkyl or alkenyl but at least one is alkenyl,

M is alkyl or X,

A is H or Alkyl

A′ is H or Alkyl,

x+y=1-30.

X=mono, di or trivalent cation, ammonium or amine,

If R and/or R″ are alkenyl they will also form the corresponding product (6) where MOOR′ is replaced by MOOR and/or MOOR″.

### FIG. 2: Reaction of Methyl Ester with Aryl Alkyl Sulfonic Acid

Sources of naturally occurring fats, glycerides, and oils include but are not restricted to palm oil, castor oil, jojoba oil, jatropha oil, tall oil, tallow, canola oil, rapeseed oil, high erucic rapeseed oil, soybean oil, sunflower oil, safflower oil, meadowfoam oil, crambe oil, fish oil, coconut oil, linseed oil, flax oil, palm kernel oil, peanut oil, tung oil and animal fats. In general any naturally occurring oil, fat or glyceride yielding unsaturated fatty acids is suitable for this process. Alcohols include but are not limited to methanol, ethanol, propanol, isopropanol, butanol, iso-butanol, sec-butanol. Alkali includes but is not limited to sodium hydroxide, sodium carbonate, potassium hydroxide, potassium carbonate, sodium methylate, potassium methylate.

The unsaturated esters or fatty acids derived from the glycerides reacts with the aryl alkyl sulfonic acid as describes in U.S. Pat. No. 6,043,391 to form a new surfactant combining both the aryl alkyl sulfonic acid and the fatty acid ester into one new anionic surfactant molecule in the acid form. This is subsequently neutralized to form the anionic surfactant in the salt form (7) of the present invention. The saturated ester components of the oil can be removed by distillation or extraction and used as bio-fuel. The new surfactant can be neutralized with a mono or divalent alkali salt or ammonia or an amine to form a neutral anionic surfactant.

The saturated fatty acid esters present in the oil do not react with the aryl alkyl sulfonic acid and they can be easily separated by distillation or extraction because of the boiling point difference with the new surfactants formed. These saturated fatty acid esters can be used as bio-fuel or solvent. They can also be left with the surfactant as solvent. The biodiesel formed can also be used to provide energy to run the manufacturing plant running the reaction.

### Example 1

This example discloses the procedure used to prepare the sulfonate surfactant of the invention from a mixture of methyl palmitate and methyl oleate. 410.2 grams (0.84 Moles) of Oil C hem Technologies XSA-1416 was added to a 1 liter three-necked flask. XSA 14-16 is depicted in structure 5 where x+y=11 to 13 and both A and A′ are CH₃. The contents were heated and held at 130-135° C. 410.2 grams (1.43 Moles) of CE-1618, a methyl oleate/methyl palmitate blend available from Proctor and Gambles Corporation containing 40 mole % methyl palmitate and 60 mole % methyl oleate, was added to the flask dropwise with stirring over a four hour period, keeping the temperature in the flask between 130-135° C. After all the methyl ester was added, the contents were mixed at 130-135° C. an additional four hours. The methyl ester content was determined by GLC and the methyl oleate was found to be completely gone while the amount of methyl palmitate remained essentially unchanged.

The methyl palmitate was removed by vacuum distillation at 150° C. leaving a viscous dark brown product. The product was neutralized to pH 9 with aqueous NaOH to bring the final activity to 50 wt %. This yielded a clear, low viscosity yellow liquid.

### Example 2

For Improved Oil Recovery applications, the surfactants produced by the present invention can be used alone or formulated with other surfactants as is known to the art, including but not limited to, anionic, amphoteric, nonionic surfactants, alkali, polymer, co-solvents and added into injection fluids and then introduced into oil bearing formations to recover residual oil by methods known to those familiar with the art. The surfactants derived from the present invention have been demonstrated to produce ultra-low interfacial tensions (IFT) between oil and aqueous brines such as water, sea-water, produced water from oil wells. Ultra-low IFT has been shown to be a necessary requirement for increasing the capillary number and for allowing the injection fluid to penetrate minute pores in oil reservoirs and displacing the entrapped oil.

The product from example 1 was diluted with 2.5% NaCl solution to a final activity of 0.1 wt %. The interfacial tension (IFT) of this solution against a 31 API Gravity crude oil was measured at 80° C. using a Grace Instruments M6500 Spinning Drop Tensiometer. The IFT found after spinning for 30 minutes was 0.00862 mN/m which indicates the material is a good candidate for oil recovery.

Further embodiments and alternative embodiments of various aspects of the present invention may be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as the presently preferred embodiment. Elements and materials may be substituted for those illustrated and described herein, parts and processes may be reversed, and certain features of the invention may be utilized independently, as would be apparent to those skilled in the art after having benefited by this description of the invention. Changes may be made in the elements described herein without departing from the spirit and scope of the invention as described in the flowing claims. In addition, it is to be understood that features described herein independently may, in certain embodiments, be combined.

What is claimed:

1. A process for conversion of naturally occurring fats, glycerides, or oils to anionic surfactants and esters by
a) transesterifying the glycerides with an alcohol,
b) reacting the esters with an arylalkyl sulfonic acid,
c) neutralizing the resulting sulfonic acid, and
d) separating the resulting anionic surfactant from the un-reacted, unsaturated salts or esters.

2. The process described in claim 1 where the naturally occurring fats, glycerides or oils are chosen from the group palm oil, castor oil, jojoba oil, jatropha oil, tall oil, tallow, canola oil, rapeseed oil, high erucic rapeseed oil, soybean oil, sunflower oil, safflower oil, meadowfoam oil, crambe oil, fish oil, coconut oil, linseed oil, flax oil, palm kernel oil, peanut oil, tung oil, animal fats.

3. The process described in claim 1 where the alcohol is chosen from the group methanol, ethanol, propanol, isopropanol, butanol, isobutanol, sec-butanol.

4. The process as described in claim 1 where the alkali is chosen from the group sodium hydroxide, sodium carbonate, potassium hydroxide, potassium carbonate, sodium methylate, potassium methylate.

5. The process for conversion of naturally occurring fats, glycerides or oils as described in claim 1 where the sulfonic acid is neutralized with a mono, di or trivalent alkali salt or ammonia or an amine.

6. The process for conversion of naturally occurring fats, glycerides or oils as described in claim 1 where the alkylaryl sulfonic acid has the structure 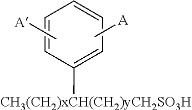
where A=H or alkyl,
A′=H or alkyl,
x+y=1-30.

7. The process for conversion of naturally occurring fats, glycerides or oils as described in claim 1 where the anionic surfactants have the structure 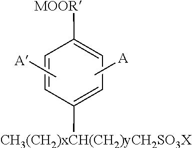
where A=H or alkyl,
A′=H or alkyl,
M=Alkyl, or X,
x+y=1-30,
X=mono. Di, trivalent cation, NH₄ or amine.

8. The process for conversion of naturally occurring fats, glycerides or oils described in claim 1 where the un-reacted salts or esters are allowed to remain with the anionic surfactant.
